SEQUENCE LISTING (15 sequences)

SEQ ID NO: 1 - DNA, 24 bp, Artificial Sequence
  human B3GALT4 sense primer for PCR
tccggagaacctgaaccagagaaa

SEQ ID NO: 2 - DNA, 24 bp, Artificial Sequence
  human B3GALT4 antisense primer for PCR
tctcccagcaagaatagcgtctgt

SEQ ID NO: 3 - DNA, 24 bp, Artificial Sequence
  human KIF1A sense primer for PCR
ttctaccacgtccagaacatcgca

SEQ ID NO: 4 - DNA, 24 bp, Artificial Sequence
  human KIF1A antisense primer for PCR
tggtagtggccaaagacctcgaaa

SEQ ID NO: 5 - DNA, 24 bp, Artificial Sequence
  human DHRS9 sense primer for PCR
acactgttagaccaagggcacaga

SEQ ID NO: 6 - DNA, 24 bp, Artificial Sequence
  human DHRS9 antisense primer for PCR
atttgatgcagcgtgtagccaagg

SEQ ID NO: 7 - DNA, 24 bp, Artificial Sequence
  human EMR2 sense primer for PCR
aacctcgaagatgggtatggcaca

SEQ ID NO: 8 - DNA, 24 bp, Artificial Sequence
  human EMR2 antisense primer for PCR
agatggttctcactctgttgccca

SEQ ID NO: 9 - DNA, 24 bp, Artificial Sequence
  human KIAA1199 sense primer for PCR
tgggtgtctgaacagctattgggt

SEQ ID NO: 10 - DNA, 24 bp, Artificial Sequence
  human KIAA1199 antisense primer for PCR
actctggtgcctcgtgcaagatta

SEQ ID NO: 11 - DNA, 1704 bp, Artificial Sequence
  human B3GALT4 sequence
gaggcgggggaggggccgtgagtgccgcagtcggccagccatggagcggagcttgctggcggcgaggccgcggcgacaaggtagccacccccgcagcatgcctcgaccgcggtccgcagctgcaccgcctctccccgccccccagggtgcgctggtcccggtcgcgcgctcagacctccgcatcccgggcgtggtcggttaagtccccggccgtgacccaggcccggggagctagtctccgcccttcgctcttacggatcccctcggagtacgccgcaccatgcagctcaggctcttccggcgcctccttctcgccgctttgctgctggtgatcgtctggaccctcttcgggccttcggggttgggggaggagctgctgagcctctcactagcctccctgctcccagcccccgcctcaccggggccgcccctggccctgccccgcctcttgatccccaaccaggaagcttgcagtggtcccggggcccctcccttcctgctcatcctggtgtgcacggctccggagaacctgaaccagagaaacgccattcgggcttcgtggggcgggctgcgcgaggcccgggggctcagggtacagacgctattcttgctgggagagccgaacgcacagcaccccgtgtggggttcccaggggagtgacctggcctcggagtcagcagcccagggggatatcttgcaggccgccttccaggactcctaccgcaacctcaccctaaagaccctcagcgggctgaactgggctgagaaacactgccccatggcccgatacgtcctcaagacggacgatgatgtgtatgtcaacgtccctgaactggtatcagagctggtcttgcgagggggccgttgggggcaatgggagagaagcacggaaccccagagagaggctgagcaggaaggaggccaggttttgcacagcgaggaagtgcctcttctgtacttgggccgggtgcactggcgcgtgaacccctctcggacaccggggggcaggcaccgcgtatcagaggagcagtggcctcacacctggggcccctttccaccctatgcctcaggcacggggtatgtgctgtcagcgtctgctgtgcagctcattctcaaggtggccagccgggcaccccttctcccattagaggatgtctttgtgggggtaagtgcccgacgaggaggcctcgccccaacacagtgtgtcaagctggctggtgccacccactacccgctagaccggtgctgctatgggaaattcctgctgacgtcccacaggctggacccctggaagatgcaggaagcctggaagctggtgggtggctctgacggggaaaggactgcgcccttttgctcctggttccagggagtcctgggcatcctgcggtgtcgagcaatagcctggcttcagagctgagagtgcctggggccacaggaaaggcaggaacaggaccttctctctcccaggcccaacgcaggggccctcactggctgcagctgatctgtttccttataccagatcctcagtctcactaaagacagcgatatgggagacacccaggggcctggcccgccagcccaaaagatggtcatcgggaagagaaaaagaaaaaaatgctgcagttgttctctcaagctagggcagaagaggggtgtcaagctcctcaataaacttgtctccacttcaaaaaaaaaaaaaaaaaa

SEQ ID NO: 12 - DNA, 8743 bp, Artificial Sequence
  human KIF1A sequence
gtgttccccccacactggggctcccactactgcgaggagtgacccacgaaggccacagagatggccggggcttcggtgaaggtggcggtgcgggtccgccccttcaattcccgggaaatgagccgtgactccaagtgcatcattcagatgtctggaagcaccaccaccattgttaaccccaaacagcccaaggagacgcccaaaagcttcagctttgactactcctactggtcgcacacctcacctgaggacatcaactacgcgtcgcagaagcaggtgtaccgggacatcggcgaggagatgctgcagcatgcctttgagggatacaacgtgtgcatcttcgcctatgggcagacgggtgccggcaagtcctacaccatgatgggcaagcaggagaaggaccagcagggcatcatcccacagctctgcgaggacctcttctctcggatcaacgacacgaccaacgacaacatgtcctactccgtggaggtcagctacatggagatttactgtgagcgcgtccgtgacctcctgaaccccaagaacaagggcaaccttcgcgtgagggagcacccactgctggggccctacgtggaggacctctccaagctggctgtcacctcctacaatgacatccaggacctcatggactcagggaacaaggccaggaccgtggcggccaccaacatgaatgagaccagcagtcgctcccacgccgtcttcaacatcatcttcacccagaagcgccatgacgcagagaccaatatcaccacggagaaggtgagcaaaatcagcctggtggacctggctgggagcgagcgggctgactccacgggagccaagggcacgcgcctcaaggagggggccaacatcaacaagtcgctgaccaccctgggcaaggtcatctccgccctggctgaaatggactccggacccaacaagaacaagaaaaagaagaagacagatttcattccgtaccgagattccgtgttgacctggctcctccgggaaaacctgggcggtaactcaaggacagctatggtggcagccttgagtcctgcagacatcaactacgatgagacccttagcacgctgaggtatgctgaccgggccaagcagatccgctgcaatgctgtcatcaatgaggaccccaacaacaagctgatccgcgagctgaaggatgaggtgacccggctgcgggaccttctgtacgcccagggtcttggcgacatcactgacatgaccaatgccctggtgggtatgagcccctcatcctcgctctcagccctgtccagccgcgcggcctccgtgtccagcctccacgagcgcatcttgtttgccccgggcagcgaggaggccattgaaagactgaaggaaacagagaagatcatagctgagctcaatgagacctgggaggagaagctgcggcggacagaagccatccggatggagagggaagccctgctggccgagatgggtgtggccatgagggaggatggcggcaccttgggcgtattctctcccaaaaagacaccacacctcgtcaacctgaacgaggacccgctgatgtctgagtgcctgctctactacatcaaggatgggatcaccagagtgggcagggaggatggcgagaggcggcaggacattgttctgagtgggcacttcatcaaggaggagcactgcgtcttccggagcgactccaggggaggcagcgaagctgtggtgaccttggagccctgtgagggggcagacacctacgtcaatggcaagaaagtcacagagcccagcatcctgcgttcaggaaaccgcatcatcatgggtaagagccatgtgttccggttcaaccaccccgagcaggcccggcaggagcgtgagcgcacgccttgtgcggagacgccagctgagcctgtggactgggccttcgcccagcgtgagctgctggagaagcagggcatcgacatgaagcaggagatggagcagaggctccaggaactggaggaccagtaccgccgcgagcgggaggaggccacctacctgctggagcagcagcggctggactatgagagcaagctggaggctctgcagaagcagatggactccaggtactacccggaggtgaacgaggaggaggaggagcccgaggatgaagtccagtggacagagcgggagtgtgagctggcgctctgggccttccggaagtggaagtggtaccagttcacgtctctgcgggacctgctgtggggcaacgccatcttcctcaaggaggccaatgccatcagcgtggagctgaaaaagaaggtacaattccagtttgtcctcctgacggacacactctactcccctctgccacccgacctgctgcccccagaggccgccaaagaccgagagacgcggcccttcccccgcaccattgtggccgtggaggtccaggaccagaagaacggggccacccactactggacgctggagaagctcaggcagcgtctggacctgatgcgggagatgtacgaccgcgctgcagaggtgccctccagtgtcatcgaggactgtgacaacgtggtgaccggcggagaccccttctatgaccgcttcccctggttccggctggtgggcagggccttcgtgtacctgagcaacctgctgtaccccgttcccctggtacaccgtgtggcaatcgtcagcgagaagggcgaggtgaagggcttcctccgcgtggccgtccaggccatctcagccgatgaagaggcccctgattatggctctggcgtccgccagtcgggaactgctaaaatctcctttgatgaccagcattttgaaaagttccagtccgagtcttgccccgtggtggggatgtcccgctcgggaacctcccaggaagagcttcgcatcgtggagggccagggccagggtgcagacgtggggccctcagccgatgaagtcaacaacaacacctgttcagcagtgcccccagaaggcctcctcctagacagctctgagaaagccgccctggatgggcccctggatgctgccctggaccacctccgcctgggcaacaccttcaccttccgtgtgacagtcctgcaggcgtccagcatctctgccgaatatgccgacatcttctgccagttcaacttcatccaccgccacgacgaggccttctccacagagcccctgaagaacacaggcagaggccccccacttggcttctaccacgtccagaacatcgcagtggaggtgaccaagtccttcattgagtacatcaagagccagcccattgttttcgaggtctttggccactaccagcagcacccgttcccgcccctctgcaaggacgtgctcagccccctgaggccctcgcgccgccacttccctcgggtcatgccactgtccaagccagtgcccgccaccaagctcagcacactgacgcggccctgtccgggaccctgccactgcaagtacgacctgctggtctacttcgagatctgtgagctggaggccaacggcgattacatcccggccgtggtggaccaccgtgggggcatgccatgcatggggaccttcctcctccaccagggcatccagcgacggattacggtgacactactgcatgagacaggcagccatatccgctggaaggaagtgcgcgagctggtcgtgggccgcatccgaaacactccagagaccgacgagtccctgatcgaccccaacatcttgtctctcaacatcctctcttccggatacatccacccagcccaagatgaccggaccttttaccaatttgaggctgcgtgggacagctccatgcacaactctctcctgctgaaccgggtcaccccttatcgagagaaaatctacatgacactctccgcttatatcgagatggagaactgcacccagccggctgttgtcaccaaggacttctgcatggtcttctattcccgtgatgccaagctgccagcctcgcgctccatccgcaacctctttggcagtgggagccttcgggcctcagagagtaaccgtgtgactggtgtgtacgagctcagcctgtgccacgtggctgacgcgggcagcccagggatgcagcgccggcgccgacgagtcctggacacatctgtggcctatgtccggggcgaggagaacctggcaggctggaggccccggagtgacagtctcattctggaccaccagtgggagctggagaagctgagcctcctgcaggaggtggagaagactaggcactacctgctcctgcgggagaagctggagaccgcccagcggcctgtcccggaggcactgtccccggccttcagcgaggactctgagtcccatggctcctccagcgcctcctccccgctctcggctgagggccgcccatcacccctggaggctcccaacgagaggcagcgggagctggccgtcaagtgcttgcgcctgctcacgcacacattcaacagagagtacacacacagccacgtctgcgtcagtgccagcgagagcaagctctccgagatgtctgtcaccctgctccgggacccgtcgatgtcccctctaggggtggccactctcaccccctcctccacttgcccctctctggttgaagggcggtacggtgccactgacctgaggaccccgcagccctgctcccggccagccagcccagagcccgagctgctgccagaggccgactccaagaagctcccttcccctgcccgggcaacagagacagacaaggagccccagcgcctgctggtccctgacatccaggagatccgagtcagcccgatcgtttccaagaaggggtacctgcacttcctggagccgcacacgtcaggctgggccaggcgcttcgtggtggtgcggcgcccctatgcctacatgtacaacagcgacaaggacaccgtggagcggttcgtgctcaacctggccactgcccaggtggagtacagtgaggaccagcaggctatgctcaagacacccaacacattcgcggtgtgcacggaacaccgcggcatcctgctgcaggccgccagcgacaaggacatgcatgactggctgtacgccttcaaccccctcctggccgggaccatacggtccaagctctccagaaggaggtctgcccagatgcgggtctgaacctgagccctcccgtgacagccggcaggcccagcccatcccctccctcatcctcgtctgtcctgtcacctgccgcccagcccctctcctgccagacagcccacgaccgggtcgaccccccaggggacgcccatgccaggcccggggacctgtgccacacgaccagctgtgctcccagcagaggctgtgcgtgtcagttcttcttgcagaatgtgctctggtggaacaagttgggagaggctgggggggccaagggcacaggttacgggggttcttgctgccgttctaatatttttttaagcatagacagacttataattaatatacgttagttagtgacattgaaacagtcaactcggaaattaactataagacttgttctatttataagtatttatttctaatgcctccacatagccctgtaatattcagatggaacccccaaccacctccaccctgtttgttcccacatgtgtctcccaagcctgctagggacaggcagggcagggacagccaccttggaaggccgcagtgaggagctgtctggaccagtggggcaccttggggctagcacacgggtgtatcgcctgggccccaggcttctccatggccacatgggtcctgggtgtatgtgtgggagagtgggggggtgtctttggtgcctgaagtctgcgcggcatggagggtggtgtgagttcctctggtgggagggagaacgcacatctcttctgggcggccacctgaggagtgactccaagaagagttccggcagctttccccaggaaagggtgaggggtgacactcggctctggctctgagatgaggcagacggcacccaggctgtgatctgtcctgggcggggaccaggagggagcggggtcgggatcacctgccagtgtgcagactctgggactgcgtgctgtctccggaccatcagggtagggtggtgggttgagaccaggaagtcagggaagatcggaattcagggcgacggtctaggtgtcgagggctgtggcgcagcctcttcagctgcggcgagaaatggagtgagtcaaggtagcttctgggaagaaatgctgccattagcaggtttcttgcaaagactttcctctctttgttcccagggcagagagtttctgtgagtcccactgagaaaatcccatggggtgggggtatcctggtcggtcggcaatggagggtggctggcttggtggttattgtcttcaaggagctcttcgctgctgcatctgcggtgtccctttgttcttgtcccatttcaccccctctgcagacaccaatgtccgagggccacccaggacaggacgggggtcagccccaagctgagagtctggtcataggagtcatgtccagaggcctagggaggttttagggccctccccacccacacccacaggtcgatttggtctctttttagctcaaggaaagacagtagccaagcaacagagcccctctcccgccgtggcccgtgggagcagttacatcgggtctggtgctccagacctagggcccagcactttcatcagatcctgcctcctggagtgggggaaacgcagcaccccactggttctgaggcccctaccctcccaggctgtcccacgtgatgctgacatgagcctcagagaccccaatcccatgcctgggggagagacagcggctcaggagtgggggagcacgggccttctacaccacatgggaaggctctggcatgaggttttcctttgggaaggttgtttgggcccctgaagttccatctccgagagtggtgtgcagggcaggccagggcccatgctggctgcagtctctgtggctgcctgcctgggccagcctgtttgggagctgggactgtgggctcgccttttcgtacctgggctcaggtgcggtgtggccaccgccaccctcatcccctgcctgggaggctctcccaggggctgatgggggggttctgtgagggagaatcagggctcgggaagccacgcctgggaaggcaggacacaggacacagcagctttctttggaaatctcccaggtgaggattcacatcccaaaataaaattcagaagccaggtggcctgtgctccccatgggtgacctctggaggcagtggaccaagatgcagcaaggagaggatgcagaacagcttcttgcagaagcacctgctccggcatccagcgctgcctggaggcaggaaggagaggcagggcaggacacgctggtctgagatgagggggagccccacgggccccaggcaggctagaggaggcacaggccctgccacggccaactcaggtcagccagcctgaggctgtggcctccaaagggtctgggcgcaccccccaggtcgcaggtgtctgaggccagccaacctgcagagcactcgcggcgtgggtgggctgagtggaggtgcctgggagctgcctaaattcagaagcctccactgccatggagactgcctggctgtgtcctctcagccaggtgccgtgttggccttggctaggacccagagccacactgcagcctccgccgactccctccttccgggcctgctctagtgaggagtgcctaagccaggacccagaaactcagagttgatggtgagaggaggccgcctgagtcaggacacctggcttcttgaaaagttcgctctgccagaaaccacctagggaccacgttagctgccttccttgagctccccaggagtcggtttccacatctgtgaggtggagggctggtgtggaggtgctcatggggtgcggtgcttgggagacagccaggcccagggtggctgctgcttcctgctaagtgggggaggtgagacagatctggaaagccgtctccctcagatggtttcatttaatgctttatactgccgagtctgggggcttgttttggtttgggggcagccatcctccaccagaaagggggagctccttccgctgcggctccagatagatggggatgccgggctccaggccgaccagcacttgggatctgatgggacacggccagtgcctaggggtgccaagtccaaggcctcccactgggagtcatcgctgagaagatgccaatgtttcatccaccggctgcacaggcacaaactcccccacccaggacggctgtgatgaggtggccctccctgtcaaccctggtccctggagtccccagcacctggggccctggtggggctgatgtcacaggtgtttactgtgctgctgcactggtcctatgccagcctcacccatgtggggaccacggaaggcacactcccttacccccggtgccgggccgtgcggtcccccagacggacagcagctgtggcgacctgcgtttctccctgggcctgtgcttcctgtagttagtccgtcccctggttcccctgtggctcagaggccgcgtccctggcttgtacatatgtgattgctgtgggcacaccccagaccccatgtcatagctgccgtcccgacgtcacgacgcctgtcccgatgtcttacacccgagtgttagccctaggctcctgtactgtgcgtgcacttgaggctctgtccaattaagaaataaatgtggctcttactcaacacagtcaaaaaaaaaa

SEQ ID NO: 13 - DNA, 2882 bp, Artificial Sequence
  human DHRS9 sequence
atctgaaaaattaataattccttaattatcaaatatccattatttaaatttataattgtgtcataaatattgtcataaatagatttgctgttttaaagcttgttccttcattttctctgttttgttttagataaacattgtcataaatagatttgttgttttaaagcttgttccttcattttctctgattgttttgttttagattcagaggttacttatgcttgtttgttacatggatgttacatgtgtaatgggggatattggacttctagtgtactcatcacccatatactgaacactgtactcaaaagggattgaaagaaactaggaaacttggcaggaagatcattcttaagccaggaaaaaaatttttaatgctcacatgtgaacatgtgatggtcataccagaaggagcacccacctccctccctctgtgacagacacattttcttagccttcacctttccttctttcaagttgctgaaaatccacagtgtttctgttcatttgttactttcattctcacctatcttctctcttgctccatctaccagaacaataattccccatataatacttctcacttcacttttcaacgcaggacctcttgttgggtctgatctgtttgtctgtccgctttatcaatattatcagatgtaagtttacatgaatacacacacatattcactaaactgaggggaaaaaatgccttgtaggtcataaaaaagcagggaaattcccaacaattcatatttgatccctggatccaggggtggcagcaataagcctgctttagatatttactccccattttatgatccggtggtttggtttttcaaatgatgatatggctcctttcgcaatgacttgatgtttaggaggtgtgcttcaataaatacattttaaaatcaacaatcaagttagagttgtacaaatggctctgaaatgtcccactacactgttagaccaagggcacagattgtgcttctgtactatttatcctagtatccctcggcatatattaactgctctaaaaatctccttggctacacgctgcatcaaatcaaagttaaatgttataccacctttctattctatttttaatattcaaagagggtgctcagattttagaacaaatttcaatgtttaagtacacacaaaaaaatcattaactcatatatttcaagagtaggaaatgggaactggtgttaaaactcttataacaaatgtcactgtcttaagggacagtgtttaaaaacgcatacctggccgggcgcggtggctcatgcctgtaatcccagcactttgggaggccgaggccggcggatcacaaggaaaacaaactcaggaagaaaaagaaaagcagaagtgatcaaggagagcgctcgagttgcaatattttcctttggctgctgacaggcagttactataaagcattgtgcatggacaccatcttcttgtattatacaagaaaggagtgtacctatcacacacagggggaaaaatgctcttttgggtgctaggcctcctaatcctctgtggttttctgtggactcgtaaaggaaaactaaagattgaagacatcactgataagtacatttttatcactggatgtgactcgggctttggaaacttggcagccagaacttttgataaaaagggatttcatgtaatcgctgcctgtctgactgaatcaggatcaacagctttaaaggcagaaacctcagagagacttcgtactgtgcttctggatgtgaccgacccagagaatgtcaagaggactgcccagtgggtgaagaaccaagttggggagaaaggtctctggggtctgatcaataatgctggtgttcccggcgtgctggctcccactgactggctgacactagaggactacagagaacctattgaagtgaacctgtttggactcatcagtgtgacactaaatatgcttcctttggtcaagaaagctcaagggagagttattaatgtctccagtgttggaggtcgccttgcaatcgttggagggggctatactccatccaaatatgcagtggaaggtttcaatgacagcttaagacgggacatgaaagcttttggtgtgcacgtctcatgcattgaaccaggattgttcaaaacaaacttggcagatccagtaaaggtaattgaaaaaaaactcgccatttgggagcagctgtctccagacatcaaacaacaatatggagaaggttacattgaaaaaagtctagacaaactgaaaggcaataaatcctatgtgaacatggacctctctccggtggtagagtgcatggaccacgctctaacaagtctcttccctaagactcattatgccgctggaaaagatgccaaaattttctggatacctctgtctcacatgccagcagctttgcaagactttttattgttgaaacagaaagcagagctggctaatcccaaggcagtgtgactcagctaaccacaaatgtctcctccaggctatgaaattggccgatttcaagaacacatctccttttcaaccccattccttatctgctccaacctggactcatttagatcgtgcttatttggattgcaaaagggagtcccaccatcgctggtggtatcccagggtccctgctcaagttttctttgaaaaggagggctggaatggtacatcacataggcaagtcctgccctgtatttaggctttgcctgcttggtgtgatgtaagggaaattgaaagacttgcccattcaaaatgatctttaccgtggcctgccccatgcttatggtccccagcatttacagtaacttgtgaatgttaagtatcatctcttatctaaatattaaaagataagtcaaacattaaaaaaaaaaaaaaaaaa

SEQ ID NO: 14 - DNA, 6483 bp, Artificial Sequence
  human EMR2 sequence
ggggggtctgctctgtgcggtgaagcttctcttcttggcacctgcctggcatcggaagagggccccttctccctccctgggcttttatgtggacactgtaatgcctcagttttctttctttctttttgtttttgacacagggtctcactctatcacccaagctggagtgcagtggcaccatcttagctcaccgcagcctcaaactccccagctcgggtgattcttcctgcctcagcctcctgagtagtagcagctgggaccacagacgtctgccaccaagcccagctgcaccgccagttccggggagggccctgggccagcggctgtccgccccccctcctttataaagtcctggcctcgggacagcccgcacagctgcccagcctgcggagacgggacagccctgtcccactcactctttcccctgctgctcctgccggcagctcagctggaaccatgggaggccgcgtctttctcgtctttctcgcattctgtgtctggctgactctgccgggagctgaaacccaggactccaggggctgtgcccggtggtgccctcaggactcctcgtgtgtcaatgccaccgcctgtcgctgcaatccagggttcagctctttttctgagatcatcaccacccccatggagacttgtgacgacatcaacgagtgtgcaacactgtcgaaagtgtcatgcggaaaattctcggactgctggaacacagaggggagctacgactgcgtgtgcagcccaggatatgagcctgtttctggggcaaaaacattcaagaatgagagcgagaacacgtgtcaagatgtggacgaatgtcagcagaacccaaggctctgtaaaagctacggcacctgcgtcaacaccctcggcagctacacgtgccagtgcctgcctggcttcaagctcaaacctgaggacccgaagctctgcacagatgtgaatgaatgcacctccggacaaaacccatgccacagctccacccactgcctcaacaacgtgggcagctatcagtgccgctgccgcccgggctggcaaccgattccggggtcccccaatggcccaaacaataccgtctgtgaagatgtggacgagtgcagctccgggcagcatcagtgtgacagctccaccgtctgcttcaacaccgtgggttcatacagctgccgctgccgcccaggctggaagcccagacacggaatcccgaataaccaaaaggacactgtctgtgaagatatgactttctccacctggaccccgccccctggagtccacagccagacgctttcccgattcttcgacaaagtccaggacctgggcagagactacaagccaggcttggccaataacaccatccagagcatcttacaggcgctggatgagctgctggaggcccctggggacctggagaccctgccccgcttacagcagcactgtgtggccagtcacctgctggatggcctagaggatgtcctcagaggcctgagcaagaacctttccaatgggctgttgaacttcagttatcctgcaggcacagaattgtccctggaggtgcagaagcaagtagacaggagtgtcaccttgagacagaatcaggcagtgatgcagctcgactggaatcaggcacagaaatctggtgacccaggcccttctgtggtgggccttgtctccattccagggatgggcaagttgctggctgaggcccctctggtcctggaacctgagaagcagatgcttctgcatgagacacaccagggcttgctgcaggacggctcccccatcctgctctcagatgtgatctctgcctttctgagcaacaacgacacccaaaacctcagctccccagttaccttcaccttctcccaccgttcagtgatcccgagacagaaggtgctctgtgtcttctgggagcatggccagaatggatgtggtcactgggccaccacaggctgcagcacaataggcaccagagacaccagcaccatctgccgttgcacccacctgagcagctttgccgtcctcatggcccactacgatgtgcaggaggaggatcccgtgctgactgtcatcacctacatggggctgagcgtctctctgctgtgcctcctcctggcggccctcacttttctcctgtgtaaagccatccagaacaccagcacctcactgcatctgcagctctcgctctgcctcttcctggcccacctcctcttcctcgtggcaattgatcaaaccggacacaaggtgctgtgctccatcatcgccggtaccttgcactatctctacctggccaccttgacctggatgctgctggaggccctgtacctcttcctcactgcacggaacctgacggtggtcaactactcaagcatcaacagattcatgaagaagctcatgttccctgtgggctacggagtcccagctgtgacagtggccatttctgcagcctccaggcctcacctttatggaacaccttcccgctgctggctccaaccagaaaagggatttatatggggcttccttggacctgtctgcgccatcttctctgtgaatttagttctctttctggtgactctctggattttgaaaaacagactctcctccctcaatagtgaagtgtccaccctccggaacacaaggatgctggcatttaaagcgacagctcagctgttcatcctgggctgcacgtggtgtctgggcatcttgcaggtgggtccggctgcccgggtcatggcctacctcttcaccatcatcaacagcctgcagggtgtcttcatcttcctggtgtactgcctcctcagccagcaggtccgggagcaatatgggaaatggtccaaagggatcaggaaattgaaaactgagtctgagatgcacacactctccagcagtgctaaggctgacacctccaaacccagcacggttaactagaaaaatcttctgaataagatcttccctctttgcccgtggaaaatctgaacaatctttgagccatctagaggggaaagaaaagactttgttctgtgtgtttcaagaaattcaccatgtcagcaatatgaaggatgttatggaaggcgtgctaggcattcaattcctgcagaaaccggaaatcttccatgccctgcaatgtgctcatcaaactctcagcatatggacggccagctgtggcccatatcttggtcactctgaagcacaatatttatgaagctatagaacgttaagacctctttcacagcctctccttcctacaaagactcctccaaatcttaaaatgaagcaggaaaacgagcctaagaggactttcataccgacaacatctgaaaggactagaatgttcacaccacgatctggatttcttaattttttgtttttgtttttgttgttctctagttctacgggtttgattatttagtcatgtgaaaaatattgattactcacacatagatcaagagagacacggctcctgccttcatggagcttttaggggaaaatgaagtggctcttgcagctagagttgactcagaagccgaaattcctagaaatcaggtttctactgctaggcaattgaagtataaactattttataaacactgtcttctttcgtcttcacaccaacatgcagaaaagtttctaatctcagatcggggatgtgcaacaaattccatttcaaaggaatgacctgcaaaactcctaaatattccaagcaaatgcccttaaccctgtctgttatctgctttccttgaacagaaattctacatgaccataaaacctcgaagatgggtatggcacagttcatgccctgtaatcctagcactttgggagggtgaggcaggaggatggctcaagcccaggagtttgagaccagtgtgggcaacagagtgagaaccatctctacccaaaaaaaaaattaaaaattagccaagcatggtgatgatataggagttaaggagaaatcatttaggcaaatagcaagggtaggaagtcctcagtaaggttttccatttaatgaaaagcagcccccaaaatcattttcttttctaacaaagaacagcctgtaaaatcgagctgcagacatagacaagcaagctggaagcttccacgggtgaatgccggcagctgtgccaataggaaaaagctacctagactaggcatgtccaaaatggcggctccaagttcccttctctttgccagccatgtgtacagtaaaaagcaggcaacatagtgtcagccaaagctcatttgcataataagattagggtggggtggccagctcacataggggtaggccctaggtaaatcagacaccgccttctcaagcctgtctataaaatctggtacactatgacgagggtcagatttcccattcagacgcccctctcccatgcaagagaaagagctgttctcctttctctttcttttgcctattaaacctctgctcctggccaggcacagtggctcacgcctataatcccagcactttgggaggctgaggtggtcagatcacctaaggtcaggagttcaagaccagcctggtcaacatggtgaaatcttgtctctagtaaaaatacaaaaatatatgaaatctcacatagatgataatattaagttccaaaagcaactcaacctggtagattctaattttttttgaggcagggtcttgctttgtcacccatgctggagtacaatggcacaaacactgctcactgcagcctcgacctcccaaggcctaagcaatcctcctgcctcagtccccctccaggtatttgaaactacaggtgtgtaccaccacaccgggctaatttttgtattttttgtagagacgtgggtctcactatgctgcccaggctcaggtcttaatctcctgagctcaggcaatccgcaggcctcagcctccctaagtgcggggattacaggcttgagccactgcacctagcctctatttgttttacaaaagagaaattgagatcctgaatgttaagtgacttgcctgaggccatcccactaacaggagccagggttaggattcaaaccccatccaactggtcccagagctggagcttcttgcactgccctacactacctaccatctccatcctctgggcacctttttataagaaccaaaacattacagagcattgctttgtcaactcagctgggaacatttcccagtgcaactcacatttttcactgctctgtgcctgtccgtataagctcaatgagtattgatttaggggctttggagaactttgaatgctaccccccaagtaaccattgttggcaacctggtacctctacttttagccatttctccttctctataaatagtgcagaagtaacccacttggtaacaggcatccttgccaagcctccaccactaggtcagtgtaagaattaaagaaagaggaaagaaacacaaaaagtggcttgatggttaagacaggtttattttagagaaaacacacctgagaggggctgctggctgaattaggttagagtcttttctacagactaagagtgtttaaggatttagggtgggagagtttcttagaggcttggactgcttctgtgttttttttgttgtgcttatatgggagggagagtggtgtgtttgcttttatacatttttctgcagctgtaggcataccccccaagtctgcttttagcttccctattttagtgcacctggagggaaaggaatgtgcttattaaggcccactgttttactggggcccattgtatgagggtgaagtttggcagttacccaagagacttttcctccaccttcctctgtgcccgagctgttttatctgcattttactgtctgctttttttggctgcttatagtttttaaaaaagtaatttccttaaatccagaaggctaaaaatgaagctgaaacttaaagtggcggtgtttgtccaaaataacggggctcctgctctgccagtcagtaccctcaagtcactcctgatcctcaacctccatgcctaaggctggttcaagagaccacataatatctgccttttattacatacatgatgggtgcatgggattctgcgtgccctttgcttgatatagactgctaaggtgagatggggaatatcagagtcagctgctgcttgaggaagcagaacacacagctggaggcttggaacatgtgggtccctatgagtgtagagcccatatccccatagagtctacctagagcaggggtcgccaaatgttttcttaaagagcctgatagtgtatatgttaggctttgtgagccaggtatttacagcaactcaattctaccactgtggtatgaaaacagctatagacaatcataaatgaatgatcatggctatgttttaataaaactttacagacactgaacttgaacttccattgtgatatgaaaacagctatagacaatcataaatgaatgatcatggctatgttttaataaaactttatggacactgagcttgaaaaaaaaaaaaaaaaaa

SEQ ID NO: 15 - DNA, 7080 bp, Artificial Sequence
  human KIAA1199 sequence
gagctagcgctcaagcagagcccagcgcggtgctatcggacagagcctggcgagcgcaagcggcgcggggagccagcggggctgagcgcggccagggtctgaacccagatttcccagactagctaccactccgcttgcccacgccccgggagctcgcggcgcctggcggtcagcgaccagacgtccggggccgctgcgctcctggcccgcgaggcgtgacactgtctcggctacagacccagagggagcacactgccaggatgggagctgctgggaggcaggacttcctcttcaaggccatgctgaccatcagctggctcactctgacctgcttccctggggccacatccacagtggctgctgggtgccctgaccagagccctgagttgcaaccctggaaccctggccatgaccaagaccaccatgtgcatatcggccagggcaagacactgctgctcacctcttctgccacggtctattccatccacatctcagagggaggcaagctggtcattaaagaccacgacgagccgattgttttgcgaacccggcacatcctgattgacaacggaggagagctgcatgctgggagtgccctctgccctttccagggcaatttcaccatcattttgtatggaagggctgatgaaggtattcagccggatccttactatggtctgaagtacattggggttggtaaaggaggcgctcttgagttgcatggacagaaaaagctctcctggacatttctgaacaagacccttcacccaggtggcatggcagaaggaggctatttttttgaaaggagctggggccaccgtggagttattgttcatgtcatcgaccccaaatcaggcacagtcatccattctgaccggtttgacacctatagatccaagaaagagagtgaacgtctggtccagtatttgaacgcggtgcccgatggcaggatcctttctgttgcagtgaatgatgaaggttctcgaaatctggatgacatggccaggaaggcgatgaccaaattgggaagcaaacacttcctgcaccttggatttagacacccttggagttttctaactgtgaaaggaaatccatcatcttcagtggaagaccatattgaatatcatggacatcgaggctctgctgctgcccgggtattcaaattgttccagacagagcatggcgaatatttcaatgtttctttgtccagtgagtgggttcaagacgtggagtggacggagtggttcgatcatgataaagtatctcagactaaaggtggggagaaaatttcagacctctggaaagctcacccaggaaaaatatgcaatcgtcccattgatatacaggccactacaatggatggagttaacctcagcaccgaggttgtctacaaaaaaggccaggattataggtttgcttgctacgaccggggcagagcctgccggagctaccgtgtacggttcctctgtgggaagcctgtgaggcccaaactcacagtcaccattgacaccaatgtgaacagcaccattctgaacttggaggataatgtacagtcatggaaacctggagataccctggtcattgccagtactgattactccatgtaccaggcagaagagttccaggtgcttccctgcagatcctgcgcccccaaccaggtcaaagtggcagggaaaccaatgtacctgcacatcggggaggagatagacggcgtggacatgcgggcggaggttgggcttctgagccggaacatcatagtgatgggggagatggaggacaaatgctacccctacagaaaccacatctgcaatttctttgacttcgatacctttgggggccacatcaagtttgctctgggatttaaggcagcacacttggagggcacggagctgaagcatatgggacagcagctggtgggtcagtacccgattcacttccacctggccggtgatgtagacgaaaggggaggttatgacccacccacatacatcagggacctctccatccatcatacattctctcgctgcgtcacagtccatggctccaatggcttgttgatcaaggacgttgtgggctataactctttgggccactgcttcttcacggaagatgggccggaggaacgcaacacttttgaccactgtcttggcctccttgtcaagtctggaaccctcctcccctcggaccgtgacagcaagatgtgcaagatgatcacagaggactcctacccggggtacatccccaagcccaggcaagactgcaatgctgtgtccaccttctggatggccaatcccaacaacaacctcatcaactgtgccgctgcaggatctgaggaaactggattttggtttatttttcaccacgtaccaacgggcccctccgtgggaatgtactccccaggttattcagagcacattccactgggaaaattctataacaaccgagcacattccaactaccgggctggcatgatcatagacaacggagtcaaaaccaccgaggcctctgccaaggacaagcggccgttcctctcaatcatctctgccagatacagccctcaccaggacgccgacccgctgaagccccgggagccggccatcatcagacacttcattgcctacaagaaccaggaccacggggcctggctgcgcggcggggatgtgtggctggacagctgccggtttgctgacaatggcattggcctgaccctggccagtggtggaaccttcccgtatgacgacggctccaagcaagagataaagaacagcttgtttgttggcgagagtggcaacgtggggacggaaatgatggacaataggatctggggccctggcggcttggaccatagcggaaggaccctccctataggccagaattttccaattagaggaattcagttatatgatggccccatcaacatccaaaactgcactttccgaaagtttgtggccctggagggccggcacaccagcgccctggccttccgcctgaataatgcctggcagagctgcccccataacaacgtgaccggcattgcctttgaggacgttccgattacttccagagtgttcttcggagagcctgggccctggttcaaccagctggacatggatggggataagacatctgtgttccatgacgtcgacggctccgtgtccgagtaccctggctcctacctcacgaagaatgacaactggctggtccggcacccagactgcatcaatgttcccgactggagaggggccatttgcagtgggtgctatgcacagatgtacattcaagcctacaagaccagtaacctgcgaatgaagatcatcaagaatgacttccccagccaccctctttacctggagggggcgctcaccaggagcacccattaccagcaataccaaccggttgtcaccctgcagaagggctacaccatccactgggaccagacggcccccgccgaactcgccatctggctcatcaacttcaacaagggcgactggatccgagtggggctctgctacccgcgaggcaccacattctccatcctctcggatgttcacaatcgcctgctgaagcaaacgtccaagacgggcgtcttcgtgaggaccttgcagatggacaaagtggagcagagctaccctggcaggagccactactactgggacgaggactcagggctgttgttcctgaagctgaaagctcagaacgagagagagaagtttgctttctgctccatgaaaggctgtgagaggataaagattaaagctctgattccaaagaacgcaggcgtcagtgactgcacagccacagcttaccccaagttcaccgagagggctgtcgtagacgtgccgatgcccaagaagctctttggttctcagctgaaaacaaaggaccatttcttggaggtgaagatggagagttccaagcagcacttcttccacctctggaacgacttcgcttacattgaagtggatgggaagaagtaccccagttcggaggatggcatccaggtggtggtgattgacgggaaccaagggcgcgtggtgagccacacgagcttcaggaactccattctgcaaggcataccatggcagcttttcaactatgtggcgaccatccctgacaattccatagtgcttatggcatcaaagggaagatacgtctccagaggcccatggaccagagtgctggaaaagcttggggcagacaggggtctcaagttgaaagagcaaatggcattcgttggcttcaaaggcagcttccggcccatctgggtgacactggacactgaggatcacaaagccaaaatcttccaagttgtgcccatccctgtggtgaagaagaagaagttgtgaggacagctgccgcccggtgccacctcgtggtagactatgacggtgactcttggcagcagaccagtgggggatggctgggtcccccagcccctgccagcagctgcctgggaaggccgtgtttcagccctgatgggccaagggaaggctatcagagaccctggtgctgccacctgcccctactcaagtgtctacctggagcccctggggcggtgctggccaatgctggaaacattcactttcctgcagcctcttgggtgcttctctcctatctgtgcctcttcagtgggggtttggggaccatatcaggagacctgggttgtgctgacagcaaagatccactttggcaggagccctgacccagctaggaggtagtctggagggctggtcattcacagatccccatggtcttcagcagacaagtgagggtggtaaatgtaggagaaagagccttggccttaaggaaatctttactcctgtaagcaagagccaacctcacaggattaggagctggggtagaactggctatccttggggaagaggcaagccctgcctctggccgtgtccacctttcaggagactttgagtggcaggtttggacttggactagatgactctcaaaggcccttttagttctgagattccagaaatctgctgcatttcacatggtacctggaacccaacagttcatggatatccactgatatccatgatgctgggtgccccagcgcacacgggatggagaggtgagaactaatgcctagcttgaggggtctgcagtccagtagggcaggcagtcaggtccatgtgcactgcaatgccaggtggagaaatcacagagaggtaaaatggaggccagtgccatttcagaggggaggctcaggaaggcttcttgcttacaggaatgaaggctgggggcattttgctggggggagatgaggcagcctctggaatggctcagggattcagccctccctgccgctgcctgctgaagctggtgactacggggtcgccctttgctcacgtctctctggcccactcatgatggagaagtgtggtcagaggggagcaatgggctttgctgcttatgagcacagaggaattcagtccccaggcagccctgcctctgactccaagagggtgaagtccacagaagtgagctcctgccttagggcctcatttgctcttcatccagggaactgagcacagggggcctccaggagaccctagatgtgctcgtactccctcggcctgggatttcagagctggaaatatagaaaatatctagcccaaagccttcattttaacagatggggaaagtgagcccccaagatgggaaagaaccacacagctaagggagggcctggggagccccaccctagcccttgctgccacaccacattgcctcaacaaccggccccagagtgcccaggcactcctgaggtagcttctggaaatggggacaagtcccctcgaaggaaaggaaatgactagagtagaatgacagctagcagatctcttccctcctgctcccagcgcacacaaacccgccctccccttggtgttggcggtccctgtggccttcactttgttcactacctgtcagcccagcctgggtgcacagtagctgcaactccccattggtgctacctggctctcctgtctctgcagctctacaggtgaggcccagcagagggagtagggctcgccatgtttctggtgagccaatttggctgatcttgggtgtctgaacagctattgggtccaccccagtccctttcagctgctgcttaatgccctgctctctccctggcccaccttatagagagcccaaagagctcctgtaagagggagaactctatctgtggtttataatcttgcacgaggcaccagagtctccctgggtcttgtgatgaactacatttatcccctttcctgccccaaccacaaactctttccttcaaagagggcctgcctggctccctccacccaactgcacccatgagactcggtccaagagtccattccccaggtgggagccaactgtcagggaggtctttcccaccaaacatctttcagctgctgggaggtgaccatagggctctgcttttaaagatatggctgcttcaaaggccagagtcacaggaaggacttcttccagggagattagtggtgatggagaggagagttaaaatgacctcatgtccttcttgtccacggttttgttgagttttcactcttctaatgcaagggtctcacactgtgaaccacttaggatgtgatcactttcaggtggccaggaatgttgaatgtctttggctcagttcatttaaaaaagatatctatttgaaagttctcagagttgtacatatgtttcacagtacaggatctgtacataaaagtttctttcctaaaccattcaccaagagccaatatctaggcattttcttggtagcacaaattttcttattgcttagaaaattgtcctccttgttatttctgtttgtaagacttaagtgagttaggtctttaaggaaagcaacgctcctctgaaatgcttgtcttttttctgttgccgaaatagctggtcctttttcgggagttagatgtatagagtgtttgtatgtaaacatttcttgtaggcatcaccatgaacaaagatatattttctatttatttattatatgtgcacttcaagaagtcactgtcagagaaataaagaattgtcttaaatgtc

(19) United States
(12) Patent Application Publication
(10) Pub. No.: US 2013/0324428 A1
(43) Pub. Date: Dec. 5, 2013

(54) SPECIFIC BIOMARKER FOR IDENTIFICATION OF EXPOSURE TO LOWER ALIPHATIC SATURATED ALDEHYDES AND THE METHOD OF IDENTIFICATION USING THE SAME

(71) Applicant: KOREA INSTITUTE OF SCIENCE AND TECHNOLOGY, Seoul (KR)

(72) Inventors: Jae Chun Ryu, Seoul (KR); Mee Song, Seoul (KR); Ji-seong Yoon, Seoul (KR); Hyo Sun Lee, Seoul (KR); Wooin Ryu, Seoul (KR); Chan Young Shin, Seoul (KR)

(73) Assignee: KOREA INSTITUTE OF SCIENCE AND TECHNOLOGY, Seoul (KR)

(21) Appl. No.: 13/689,137

(22) Filed: Nov. 29, 2012

(30) Foreign Application Priority Data
May 29, 2012 (KR) 10-2012-0056716

(51) Int. Cl. C12Q 1/68

(52) U.S. Cl. CPC C12Q 1/6876
USPC 506/9; 435/6.12; 435/6.11; 435/7.92

(57) ABSTRACT

The present invention relates to a biomarker for the identification of specific exposure to lower aliphatic saturated aldehydes which are volatile organic compounds exposed in the environment, and a method for the identification of specific exposure to lower aliphatic saturated aldehydes using the same, precisely a biomarker which is up- or down-regulated specifically by lower aliphatic saturated aldehyde exposure and a method for the identification of specific exposure to lower aliphatic saturated aldehydes using the biomarker. The biomarker of the present invention is the reacted genes selected by using DNA microarray chip, which can be effectively used for the monitoring and evaluation of lower aliphatic saturated aldehyde contamination in the environment samples and at the same time as a tool for the investigation of the toxic mechanism induced specifically by lower aliphatic saturated aldehydes.

### CROSS-REFERENCES TO RELATED APPLICATION

This patent application claims the benefit of priority under 35 U.S.C. §119 from Korean Patent Application Nos. 10-2012-0056716 filed on May 29, 2012 the contents of which are incorporated herein by reference.

### BACKGROUND OF THE INVENTION

1. Field of the Invention

The present invention relates to a specific biomarker for the identification of exposure to lower aliphatic saturated aldehydes and a method for the identification of such exposure using the same, more precisely a biomarker which is specifically up-regulated or down-regulated by lower aliphatic saturated aldehydes and a method for the identification of specific exposure to lower aliphatic saturated aldehydes using the said biomarker.

2. Description of the Related Art

Aldehydes are produced by incomplete combustion of hydrocarbon or other organic materials. Aldehydes can also be produced by photochemical reaction of nitrogen oxide and some hydrocarbons, suggesting that aldehydes can be generated by pollutants in the air. Aldehydes spread in the air can produce oxidants such as peroxides and ozone and carbon monoxide via photochemical reaction of itself.

The major cause of indoor aldehyde production can be furniture, carpet, resin plywood, fabrics, paint, etc. Various aldehydes are even included in the smoke generated during cooking. Smoking is another cause to produce various aldehydes, which is also a major cause of indoor aldehyde production (Crit Rev Toxicol 35(7):609-662, 2005).

Among many aldehydes, lower aliphatic saturated aldehydes have C₀˜C₉ carbon chain. Each of them is gas or liquid having pungent odor and dissolved in water. Among them, aldehydes having C₆˜C₉ carbon chain are mainly used as flavorings or food additives, or used in perfume industry. The indoor or outdoor concentrations of lower aliphatic saturated aldehydes have not been fully investigated since many kinds of aldehydes (formaldehyde, Acetaldehyde, propionaldehyde, butylaldehyde, valeraldehyde, hexanal, heptanal, octanal, and nonanal) except formaldehyde and acetaldehyde have not been classified as hazardous materials or targets for regulation, yet. According to the previous reports rarely made, the exposure level of lower aliphatic saturated aldehydes has not been regular. For example, the exposure level in indoor environment was varied from conditions of the building and surrounding environment. Only Korea and Japan have the regulation on lower aliphatic saturated aldehydes, which is exemplified by propionaldehyde, butylaldehyde, and pentylaldehyde (only three of them).

Once exposed to the low concentration of lower aliphatic saturated aldehyde, such symptoms as ocular irritation and respiratory irritation are developed. When high concentration of the lower aliphatic saturated aldehyde is inhaled, respiratory system is irritated, resulting in such symptoms as burning feeling, nausea, dizziness, cough, phlegm, laryngitis, headache, respiratory rate increase, and dyspnea. When the said aldehyde is inhaled for a long term, convulsion, seizure, bronchitis, pneumonia, and laryngeal edema can be developed (http://toxnet.nlm.nih.gov).

The volatile organic compounds flowing through bloodstream affect the lung by diffusion of lung sac membrane. Hexane, methylpentan, isopropene, and benzene have been used as markers for the respiratory diseases (J Vet Sci 5(1):11-18, 2004). Recently, a simple diagnostic method for lung cancer has been developed based on the results of exhaled breath analysis on volatile organic compounds. Among the volatile organic compounds, aldehydes are the most representative materials commonly found in lung cancer patients (J Chromatogr B Analyt Technol Biomed Life Sci. 878(27):2643-2651, 2010). Therefore, the lower aliphatic saturated aldehyde specific biomarker can be effectively used for the screening of exposure to lower aliphatic saturated aldehyde in the environment along with the screening of pulmonary disease.

Despite the hazard in human, risk assessment data of lower aliphatic saturated aldehydes are not enough and the methods for the screening of the lower aliphatic saturated aldehydes are limited to a few classical methods such as GC-MS (Gas Chromatography-Mass Spectrometer) or HPLC (High Performance Liquid Chromatography). GC-MS or HPLC enables quantitative analysis but proper conditions have to be set up first and expensive equipments are required for the analysis. Therefore, it is important to establish molecular index for the screening of toxicity and specific gene expression in human via faster and simpler methods such as real-time RT-PCR (real-time reverse transcript polymerase chain reaction) using primers or DNA microarray chip for the fast risk assessment, and to control and manage lower aliphatic saturated aldehyde exposure.

Genome sequencing project has been completed with 6 species of mammals and 292 species of microorganisms, which has been reported to NCBI (National Center for Biotechnology Information). Based on the huge amount of data obtained thereby, genome-wide expression has been studied to understand the functions of genes. DNA microarray analysis has been performed to analyze thousands of genes at a time (Proc. Natl. Acad. Sci. USA 93:10614-10619, 1996).

Microarray indicates the glass board on which many sets of cDNA (complementary DNA) or 20-25 base pair long oligonucleotides are integrated. CDNA microarray is now produced by ink jetting or by fixing cDNA mechanically on the chip in laboratories of schools or companies including Agilent and Genomic Solutions, etc. (J. Am. Acad. Dermatol. 51:681-692, 2004). Oligonucleotide microarray is produced by direct synthesis on the chip using photolithography by Affymetrix Co., or via fixation of synthesized oligonucleotides by Agilent Co. (J. Am. Acad. Dermatol. 51:681-692, 2004).

To analyze gene expression, RNA is first extracted from tissues or cells, followed by hybridization with oligonucleotides on DNA microarray. The obtained RNA is labeled with fluorescein or isotope, which is then converted into cDNA. In oligo microarray, each of the control group and the experimental group is labeled with different fluorescent materials (ex: Cye3 and Cye 5) but hybridization is induced on the same chip simultaneously. Optical image is scanned to measure fluorescence signal. Gene expression is determined by comparing the two different fluorescence signals (Genomics Proteomics I: 1-10, 2002). The cooperation with toxicogenomics, the most recent technology using DNA microarray, enables high throughput quantitative analysis and expression pattern analysis of genes expressed in a specific tissue or cell line triggered by every chemical including not only drugs and new drug candidates but also representative environmental contaminants. Thus, specific genes that are involved in side effects of drugs and adverse actions of environmental contaminants can be identified by analyzing specific gene expression in specific cells. Accordingly, adverse actions of environmental contaminants and molecular mechanisms related to functions and side effects of drugs can be understood and further screening and identification of such material that causes toxicity and side effects can be achieved.

The present inventors observed and analyzed gene expression profiles affected by lower aliphatic saturated aldehydes in A459 cell line, the human lung cancer tissue derived cell line, by using oligomicroarray on which 42,000 human genes are integrated. As a result, the present inventors completed this invention by establishing a biomarker that is able to detect lower aliphatic saturated aldehydes specifically by using a gene up-regulated or down-regulated specifically by lower aliphatic saturated aldehydes among many other environmental materials and a method for the identification of specific exposure to lower aliphatic saturated aldehydes using the said biomarker.

### SUMMARY OF THE INVENTION

It is an object of the present invention to provide a biomarker that is up-regulated or down-regulated specifically by lower aliphatic saturated aldehyde exposure and a method for the identification of specific exposure to lower aliphatic saturated aldehydes using the said biomarker.

To achieve the above object, the present invention provides a biomarker for the identification of lower aliphatic saturated aldehyde specific exposure whose expression is specifically changed by the exposure to 6 kinds of lower aliphatic saturated aldehydes (propionaldehyde, butylaldehyde, valeraldehyde, hexanal, heptanal, and octanal).

The present invention also provides a DNA microarray chip for the identification of lower aliphatic saturated aldehyde specific exposure, on which nucleic acid sequences or their complementary strand molecules of one or more genes selected from the below group are integrated:

Genebank accession number NM_{—}003782 (B3GALT4, UDP-Gal:betaGlcNAc beta 1,3-galactosyltransferase, polypeptide 4; SEQ. ID. NO: 11), Genebank accession number NM_{—}004321 (KIF1A, kinesin family member 1A; SEQ. ID. NO: 12), Genebank accession number NM_{—}005771 (DHRS9, dehydrogenase/reductase (SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15).

The present invention further provides a method for the identification of exposure to lower aliphatic saturated aldehydes (propionaldehyde, butylaldehyde, valeraldehyde, hexanal, heptanal, and octanal) comprising the following steps:

1) measuring expression levels of genes of:

Genebank accession number NM_{—}003782 (B3GALT4, UDP-Gal:betaGlcNAc beta 1,3-galactosyltransferase, polypeptide 4; SEQ. ID. NO: 11), Genebank accession number NM_{—}004321 (KIF1A, kinesin family member 1A; SEQ. ID. NO: 12), Genebank accession number NM_{—}005771 (DHRS9, dehydrogenase/reductase (SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15), on somatic cells separated from both an experimental group suspected with lower aliphatic saturated aldehydes exposure and a normal control group;

2) screening a subject with increased or decreased expression level by comparing the expression level of the experimental group of step 1) with that of the control group; and

3) determining the screened object of step 2) to be exposed to lower aliphatic saturated aldehydes

The present invention further provides a method for the identification of exposure to lower aliphatic saturated aldehydes comprising the following steps:

1) extracting RNA from somatic cells obtained from both the experimental group highly suspected with lower aliphatic saturated aldehyde exposure and the normal control group;

2) synthesizing cDNA from the RNA extracted from both the experimental group and the control group of step 1), followed by labeling with different fluorescent materials;

3) hybridizing each cDNA labeled with different fluorescent materials of step 2) with the DNA microarray chip of the present invention;

4) analyzing the reacted DNA microarray chip; and

5) confirming the exposure to lower aliphatic saturated aldehydes by comparing the expressions of the genes integrated on the DNA microarray chip of the present invention with those of the control based on the data analyzed.

The present invention also provides a method for the identification of exposure to lower aliphatic saturated aldehydes comprising the following steps:

1) extracting RNA from somatic cells obtained from both the experimental group highly suspected with lower aliphatic saturated aldehyde exposure and the normal control group;

2) performing real-time RT-PCR (real-time reverse transcript polymerase chain reaction) with the obtained RNA using the primer sets complementary to the below genes and able to amplify them as well:

Genebank accession number NM_{—}003782 (B3GALT4, UDP-Gal:betaGlcNAc beta 1,3-galactosyltransferase, polypeptide 4; SEQ. ID. NO: 11), Genebank accession number NM_{—}004321 (KIF1A, kinesin family member 1A; SEQ. ID. NO: 12), Genebank accession number NM_{—}005771 (DHRS9, dehydrogenase/reductase (SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15); and

3) confirming the expression by comparing the gene product obtained in step 2) with that of the control.

The present invention also provides a kit for the identification of exposure to lower aliphatic saturated aldehydes comprising the DNA microarray chip of the present invention.

In addition, the present invention provides a kit for the identification of exposure to lower aliphatic saturated aldehydes comprising the primer set that is complementary to each of the below genes and is able to amplify each of them as well:

Genebank accession number NM_{—}003782 (B3GALT4, UDP-Gal:betaGlcNAc beta 1,3-galactosyltransferase, polypeptide 4; SEQ. ID. NO: 11), Genebank accession number NM_{—}004321 (KIF1A, kinesin family member 1A; SEQ. ID. NO: 12), Genebank accession number NM_{—}005771 (DHRS9, dehydrogenase/reductase (SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15).

### ADVANTAGEOUS EFFECT

As explained hereinbefore, the specific biomarker for the identification of exposure to lower aliphatic saturated aldehydes and the method for the identification of such exposure using the same of the present invention are very useful for the monitoring lower aliphatic saturated aldehydes using the reactive gene selected by using DNA microarray chip as a biomarker and for the risk assessment and also as a tool to explain the mechanism of lower aliphatic saturated aldehyde specific toxicity.

### BRIEF DESCRIPTION OF THE DRAWINGS

The application of the preferred embodiments of the present invention is best understood with reference to the accompanying drawings, wherein:

FIG. 1 is a set of graphs illustrating the cytotoxicity of 6 kinds of lower aliphatic saturated aldehydes in the human lung cancer tissue derived cell line.

FIG. 2 is a diagram illustrating the result of the gene expression analysis with the human lung cancer tissue derived cell line treated with lower aliphatic saturated aldehydes by using microarray chip

FIG. 3 is a set of graphs illustrating the comparison of gene expression profiles obtained by treating 6 kinds of lower aliphatic saturated aldehydes to select those genes showing up- or down-regulation specifically by lower aliphatic saturated aldehydes using microarray chip and real-time RT-PCR (reverse transcript polymerase chain reaction).

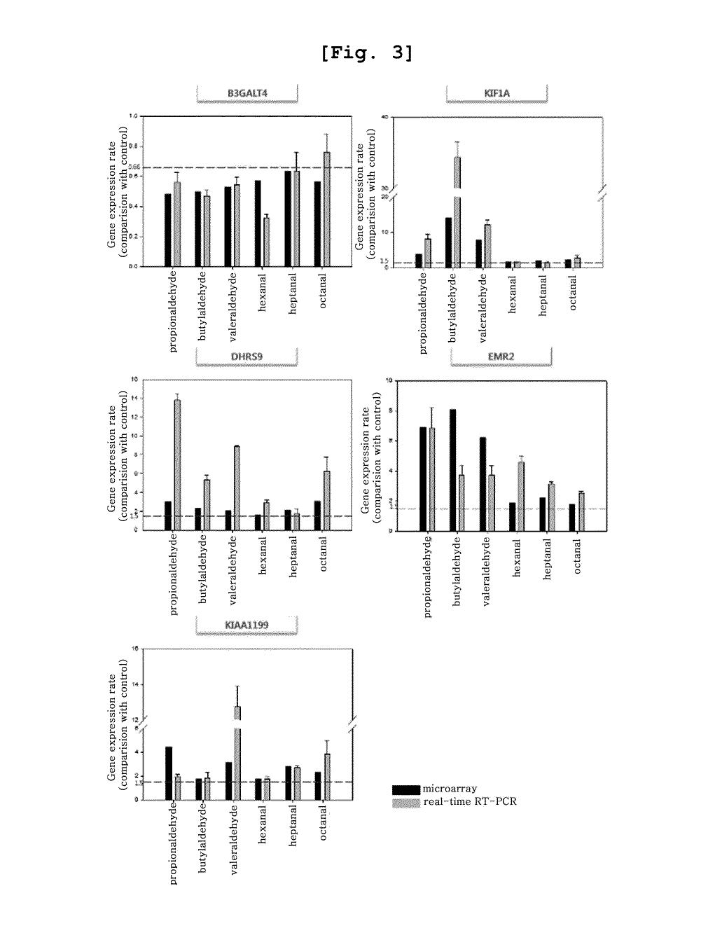

### DESCRIPTION OF THE PREFERRED EMBODIMENTS

Hereinafter, the present invention is described in detail.

The present invention provides a biomarker for the identification of lower aliphatic saturated aldehyde specific exposure whose expression is specifically changed by exposure to lower aliphatic saturated aldehydes.

The said biomarker is the genes whose expressions are 1.5 fold increased or decreased by lower aliphatic saturated aldehydes and the biomarker is composed of 5 kinds of genes whose expressions are specifically changed by lower aliphatic saturated aldehydes.

The biomarker whose expression is specifically changed by exposure to lower aliphatic saturated aldehydes is preferably selected from the group composed of as followings, but not always limited thereto:

Genebank accession number NM_{—}003782 (B3GALT4, UDP-Gal:betaGlcNAc beta 1,3-galactosyltransferase, polypeptide 4; SEQ. ID. NO: 11), Genebank accession number NM_{—}004321 (KIF1A, kinesin family member 1A; SEQ. ID. NO: 12), Genebank accession number NM_{—}005771 (DHRS9, dehydrogenase/reductase

(SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15).

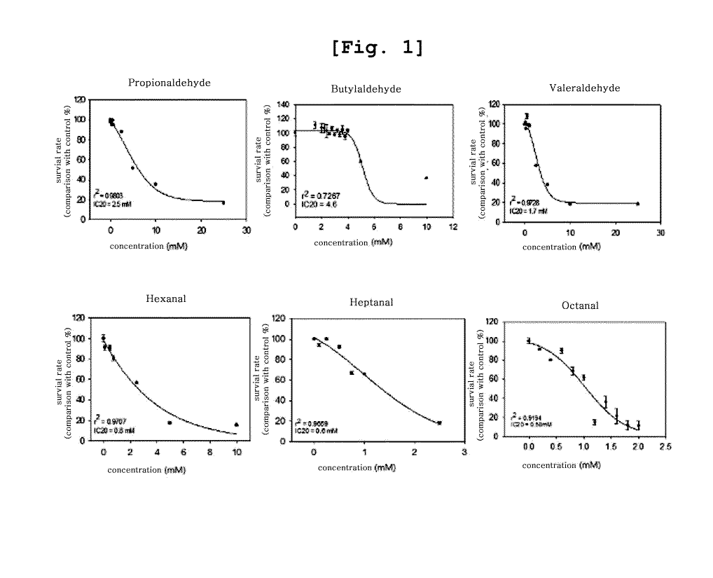

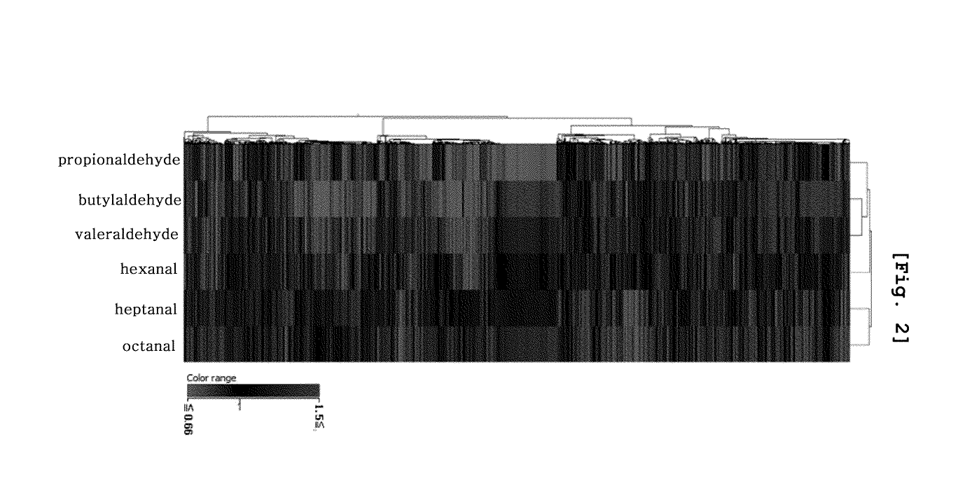

In a preferred embodiment of the present invention, to screen a biomarker for the identification of lower aliphatic saturated aldehyde specific exposure, the present inventors treated 6 kinds of lower aliphatic saturated aldehydes (propionaldehyde, butylaldehyde, valeraldehyde, hexanal, heptanal, and octanal) to the human lung cancer tissue derived cell line A549 and then investigated cytotoxicity therein. As a result, it was confirmed that the said lower aliphatic saturated aldehydes had cytotoxicity in the human lung cancer tissue derived cell line (see FIG. 1). Based on the result of that experiment, the concentrations of lower aliphatic saturated aldehydes were determined. Lower aliphatic saturated aldehydes were treated to the human lung cancer tissue derived cell line at the determined concentrations. From the cell line treated with lower aliphatic saturated aldehydes, mRNA was extracted, followed by synthesis of cDNA which was labeled with fluorescein (Cy5). The control not treated with lower aliphatic saturated aldehydes was labeled with Cy3. The fluorescein-labeled cDNA was hybridized with 8×60 k oligomicroarray chip [Human whole genome oligo microarray (Agilent, USA)], followed by scanning fluorescence image to analyze gene expression patterns (see FIG. 2). When the ratio of Cy5 to Cy3 was higher than 1.5, the gene was regarded as the one whose expression is increased. When the ratio was lower than 0.66, the gene was regarded as the one whose expression is decreased. From the result of the analysis, it was confirmed that the gene whose expression was increased took 0.04% (16 genes out of 42,405) and the gene whose expression was decreased took 0.01% (4 genes out of 42,405). Among them, 5 kinds of genes whose expressions were increased or decreased specifically by lower aliphatic saturated aldehydes were selected by using real-time quantitative PCR (see Table 4). These genes were reported to be involved in the development of lung toxicity related diseases triggered by other chemicals in previous studies, but there have been no reports saying that the treatment of lower aliphatic saturated aldehydes on them causes toxicity in human lung cancer tissue derived cells.

The present invention also provides a DNA microarray chip for the identification of lower aliphatic saturated aldehyde specific exposure, on which nucleic acid sequences or their complementary strand molecules of one or more genes selected from the below group are integrated:

Genebank accession number NM_{—}003782 (B3GALT4, UDP-Gal:betaGlcNAc beta 1,3-galactosyltransferase, polypeptide 4; SEQ. ID. NO: 11), Genebank accession number NM_{—}004321 (KIF1A, kinesin family member 1A; SEQ. ID. NO: 12), Genebank accession number NM_{—}005771 (DHRS9, dehydrogenase/reductase

(SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15).

The DNA microarray chip for the identification of lower aliphatic saturated aldehyde specific exposure of the present invention can be prepared by the method well known to those in the art. Precisely, the method for the preparation of the said microarray chip is as follows. To fix the screened biomarker on DNA chip board using as a probe DNA molecule, micropipetting based on piezolelectric method or pin spotter is preferably used, but not always limited thereto. In a preferred embodiment of the present invention, pin-spotter microarray was used. The DNA microarray chip board is preferably coated with one of active groups selected from the group consisting of amino-silane, poly-L-lysine, and aldehyde, but not always limited thereto. The board is also selected from the group consisting of slide glass, plastic, metal, silicon, nylon membrane, and nitrocellulose membrane, but not always limited thereto. In a preferred embodiment of the present invention, amino-silane coated slide glass was used as the board.

The present invention also provides a method for the identification of lower aliphatic saturated aldehyde specific exposure using the biomarker of the present invention.

The present invention provides a method for the identification of exposure to lower aliphatic saturated aldehydes (propionaldehyde, butylaldehyde, valeraldehyde, hexanal, heptanal, and octanal) comprising the following steps:

1) measuring expression levels of genes of:

Genebank accession number NM_{—}003782 (B3GALT4, UDP-Gal:betaGlcNAc beta 1,3-galactosyltransferase, polypeptide 4; SEQ. ID. NO: 11), Genebank accession number NM_{—}004321 (KIF1A, kinesin family member 1A; SEQ. ID. NO: 12), Genebank accession number NM_{—}005771 (DHRS9, dehydrogenase/reductase (SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15), on somatic cells separated from both an experimental group suspected with lower aliphatic saturated aldehydes exposure and a normal control group;

2) screening a subject with increased or decreased expression level by comparing the expression level of the experimental group of step 1) with that of the control group; and

3) determining the screened object of step 2) to be exposed to lower aliphatic saturated aldehydes.

In this identification method, the somatic cell of step 1) is preferably A549, the human lung cancer tissue derived cell line, but not always limited thereto, and any human lung cell or human lung cancer cell and tissue derived cell can be used.

In the identification method, the comparing the expression level in step 1) is performed at the level gene or protein. At this time. The gene level can be performed by RT-PCR, competitive RT-PCR, real-time RT-PCR, RNase protection assay, Northern blotting, and DNA chip. AND, the protein level can be performed by microarray or ELISA.

In the identification method, the expression level of the genes of Genebank accession number NM_{—}004321 (KIF1A, kinesin family member 1A; SEQ. ID. NO: 12), Genebank accession number NM_{—}005771 (DHRS9, dehydrogenase/reductase (SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15) is up-regulated when exposed to lower aliphatic saturated aldehydes.

In the identification method, the expression level of the gene of Genebank accession number NM_{—}003782 (B3GALT4, UDP-Gal:betaGlcNAc beta 1,3-galactosyltransferase, polypeptide 4; SEQ. ID. NO: 11) is down-regulated when exposed to lower aliphatic saturated aldehydes.

The present invention provides a method for the identification of specific exposure to lower aliphatic saturated aldehydes comprising the following steps:

1) extracting RNA from somatic cells obtained from both the experimental group highly suspected with lower aliphatic saturated aldehyde exposure and the normal control group;

2) synthesizing cDNA from the RNA extracted from both the experimental group and the control group of step 1), followed by labeling with different fluorescent materials;

3) hybridizing each cDNA labeled with different fluorescent materials of step 2) with the DNA microarray chip of the present invention;

4) analyzing the reacted DNA microarray chip; and

5) confirming the exposure by comparing the expressions of the genes integrated on the DNA microarray chip of the present invention with those of the control based on the data analyzed.

In this identification method, the somatic cell of step 1) is preferably A549, the human lung cancer tissue derived cell line, but not always limited thereto, and any human lung cell or human lung cancer cell and tissue derived cell can be used.

In the identification method, the fluorescent material of step 3) is preferably selected from the group consisting of Cy3, Cy5, poly L-lysine-fluorescein isothiocyanate (FITC), rhodamine-B-isothiocyanate (RITC), and rhodamine, but not always limited thereto, and any fluorescent material that is well known to those in the art can be used.

In the identification method of the present invention, the DNA microarray chip of step 4) is preferably whole human genome oligo microarray chip (Agilent, USA), but not always limited thereto, and any microarray chip loaded with gene demonstrating up- or down-regulation (see Table 4), among human genome, can be used. The DNA microarray chip constructed by the present inventors is more preferred. In the analyzing method of step 4), Agilent Feature Extraction 10.7.3.1 (Agilent technologies, CA, USA), or Agilent GeneSpring GX 11.5.1 (Agilent technologies, CA, USA) is preferably used, but not always limited thereto, and any software for such analysis known to those in the art can be used.

The present invention also provides a method for the identification of exposure to lower aliphatic saturated aldehydes comprising the following steps:

1) extracting RNA from somatic cells obtained from both the experimental group highly suspected with lower aliphatic saturated aldehyde exposure and the normal control group;

2) performing real-time RT-PCR (real-time reverse transcript polymerase chain reaction) with the obtained RNA using the primer sets complementary to the below genes and able to amplify them as well:

Genebank accession number NM_{—}003782 (B3GALT4, UDP-Gal:betaGlcNAc beta 1,3-galactosyltransferase, polypeptide 4; SEQ. ID. NO: 11), Genebank accession number NM_{—}004321 (KIF1A, kinesin family member 1A; SEQ. ID. NO: 12), Genebank accession number NM_{—}005771 (DHRS9, dehydrogenase/reductase (SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15); and 3) confirming the expression by comparing the gene product obtained in step 2) with that of the control.

The primer set of step 2) is preferably composed of the forward primer and the reverse primer in the length of 18-30 mer to amplify the gene of step 2), and more preferably selected from the group consisting of the following primer set 1˜primer set 5, but not always limited thereto:

Primer set 1: forward primer represented by SEQ. ID. NO: 1 and reverse primer represented by SEQ. ID. NO: 2;

Primer set 2: forward primer represented by SEQ. ID. NO: 3 and reverse primer represented by SEQ. ID. NO: 4; Primer set 3: forward primer represented by SEQ. ID. NO: 5 and reverse primer represented by SEQ. ID. NO: 6;

Primer set 4: forward primer represented by SEQ. ID. NO: 7 and reverse primer represented by SEQ. ID. NO: 8; and

Primer set 5: forward primer represented by SEQ. ID. NO: 9 and reverse primer represented by SEQ. ID. NO: 10.

Therefore, the biomarker of the present invention can be effectively used for the monitoring and evaluation of lower aliphatic saturated aldehyde contamination in environmental examples because the expression of the marker is specifically increased or decreased by lower aliphatic saturated aldehydes.

The present invention also provides a kit for the identification of specific exposure to lower aliphatic saturated aldehydes comprising the DNA microarray chip constructed in this invention.

The kit preferably contains human somatic cells additionally, but not always limited thereto.

The said human somatic cell is preferably A549, but not always limited thereto, and any human lung cell or human lung cancer cell and tissue derived cell can be used.

The kit can additionally include fluorescent material which is preferably selected from the group consisting of streptavidin-like phosphatase conjugate, chemifluorescence, and chemiluminescent, but not always limited thereto. In a preferred embodiment of the present invention, Cy3 and Cy5 were used.

The kit can additionally include reaction reagent which is exemplified by buffer used for hybridization, reverse transcriptase for cDNA synthesis from RNA, cNTPs and rNTP (premix typr or separately supplied type), labeling reagent such as chemical inducer of fluorescent dye, and washing buffer, but not always limited thereto, and any reaction reagent required for DNA microarray chip hybridization known to those in the art can be included.

The biomarker of the present invention is up-regulated or down-regulated specifically by lower aliphatic saturated aldehydes, so that it can be effectively used for the monitoring and evaluation of lower aliphatic saturated aldehyde contamination in the environment samples and as a tool to explain the mechanism of lower aliphatic saturated aldehyde specific toxicity.

In addition, the present invention provides a kit for the identification of exposure to lower aliphatic saturated aldehydes comprising the primer set that is complementary to each of the below genes and is able to amplify each of them as well:

Genebank accession number NM_{—}003782 (B3GALT4, UDP-Gal:betaGlcNAc beta 1,3-galactosyltransferase, polypeptide 4; SEQ. ID. NO: 11), Genebank accession number NM_{—}004321 (KIF1A, kinesin family member 1A; SEQ. ID. NO: 12), Genebank accession number NM_{—}005771 (DHRS9, dehydrogenase/reductase (SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15).

The primer set included in the said kit is preferably selected from the group consisting of the following primer set 1˜primer set 5, but not always limited thereto. Any forward primer and reverse primer set in the length of 15˜50 mer, more preferably in the length of 15˜30 mer, and most preferably in the length of 18˜25 mer to produce the amplified product of the biomarker gene to be 100˜300 bp long can be used.

Primer set 1: forward primer represented by SEQ. ID. NO: 1 and reverse primer represented by SEQ. ID. NO: 2;

Primer set 2: forward primer represented by SEQ. ID. NO: 3 and reverse primer represented by SEQ. ID. NO: 4; Primer set 3: forward primer represented by SEQ. ID. NO: 5 and reverse primer represented by SEQ. ID. NO: 6;

Primer set 4: forward primer represented by SEQ. ID. NO: 7 and reverse primer represented by SEQ. ID. NO: 8; and

Primer set 5: forward primer represented by SEQ. ID. NO: 9 and reverse primer represented by SEQ. ID. NO: 10.

The kit for the identification preferably contains human somatic cells additionally, but not always limited thereto.

The said human somatic cell is preferably A549, but not always limited thereto, and any human lung cell or human lung cancer cell and tissue derived cell can be used.

The kit can additionally include reaction reagent which is exemplified by reverse transcriptase for cDNA synthesis from RNA, cNTPs and rNTP (premix type or separately supplied type), labeling reagent such as chemical inducer of fluorescent dye, and washing buffer, but not always limited thereto, and any reaction reagent required for RT-PCR known to those in the art can be included.

Practical and presently preferred embodiments of the present invention are illustrative as shown in the following Examples, Experimental Examples and Manufacturing Examples.

However, it will be appreciated that those skilled in the art, on consideration of this disclosure, may make modifications and improvements within the spirit and scope of the present invention.

### Example 1

### Cell Culture and Chemical Treatment

### <1-1> Cell Culture

A549 cells (Korean Cell Line Bank), the human lung cancer tissue derived cell line, were cultured in 100 mm dish containing RPMI (Gibro-BRL, USA) supplemented with 10% FBS until the confluency reached 80%. The present inventors selected 6 kinds of lower aliphatic saturated aldehydes (propionaldehyde, butylaldehyde, valeraldehyde, hexanal, heptanal, and octanal) among many volatile organic compounds exposed in environment based on the previous studies and reports and then dissolved in DMSO (dimethyl sulfoxide). The concentration of vehicle was up to 0.1% in every experiment.

### <1-2> Cytotoxicity Test (MTT Assay) and Chemical Treatment

MTT assay was performed with A549 cell line according to the method of Mossman, et al (J. Immunol. Methods, 65, 55-63, 1983).

Particularly, the cells were distributed in 24-well plate (3.5×10⁴ cells/well) containing RPMI (Gibro-BRL, USA) and then treated with lower aliphatic saturated aldehydes dissolved in DMSO. 48 hours later, 5 mg/ml of MTT (3-4,5-dimethylthiazol-2,5-diphenyltetra zolium bromide) was added thereto, followed by culture at 37° C. for 3 hours. Then, the medium was discarded and the formed formazan crystal was dissolved in 500 μl of DMSO, which was aliquoted in 96-well plate. OD₅₄₀ was measured.

As a result, as shown in FIG. 1, IC₂₀ (the concentration showing 20% survival rate) values of lower aliphatic saturated aldehydes were as follows: propionaldehyde 2.5 mM, butylaldehyde 4.6 mM, valeraldehyde 1.7 mM, hexanal 0.8 mM, heptanal 0.6 mM, and octanal 0.58 mM. Based on this result, the following microarray experiment was performed (FIG. 1).

### Example 2

### Microarray Experiment

### <2-1> Separation of Target RNA and Fluorescein Labeling

A549 cells were distributed in 6-well plate at the density of 25×10⁴ cells/ml, to which lower aliphatic saturated aldehydes were treated for 48 hours at the concentrations determined in Example <1-2>. Total RNA was extracted from the cells by using trizol reagent according to the manufacturer's protocol (Invitrogen life technologies, USA), followed by purification by using RNease mini kit (Qiagen, USA). Genomic DNA was eliminated by using RNase-free DNase set (Qiagen, USA) during the RNA purification. The amount of total RNA was measured with spectrophotometer, and the concentration was confirmed by ND-1000 Spectrophotometer (Thermo Fisher Scientific Inc., USA) and Agilent 2100 Bioanalyzer (Agilent).

<2-2> Preparation of Labeled cDNA

For oligomicroarray analysis, cDNA was synthesized by using the total RNA obtained from the experimental group treated with lower aliphatic saturated aldehydes prepared in Example <2-1>. 30 μg of the obtained total RNA and 2 μg of oilgo (dT) primer (1 μg/μl) were mixed together, followed by reaction at 65° C. for 10 minutes, which was transferred into ice for annealing. Upon completion of annealing, reagents were added thereto as shown in Table 1 to induce reverse transcription of the annealed RNA. The total RNA extracted from the control group A549 cells was labeled with Cy3-dUTP (green). The total RNA extracted from the experimental group A549 cells treated with lower aliphatic saturated aldehydes was labeled with Cy5-dUTP (red). At that time, the two samples were mixed and purified by using Microcon YM-30 column (Millipore, USA).

TABLE 1

| Composition | Volume (μl) |
|---|---|
| 5X first strand buffer | 6 |
| dNTPs | 0.6 |
| 0.1M DDT | 3 |
| SuperScript II enzyme | 3 |
| Cy-3 or Cy-5 dUTP | 2 |

### <2-3> Hybridization

Hybridization and washing processes were performed according to the protocol provided by Ebiogen Inc.

Particularly, transcription master-mix was prepared as shown in Table 2, followed by reaction at 40° C. for 2 hours. Labeled cRNA was purified. 600 ng of the purified cRNA was reacted at 60° C. for 30 minutes for fragmentation. The prepared cRNA was mixed with 2×GEx Hybridization Buffer HI-RPM. After well mixing, the mixture was loaded on chip, followed by hybridization in the oven at 65° C. for 17 hours. 17 hours later, the chip was washed with GE Wash Buffer 1 for minute and with GE Wash Buffer 2 for 1 minute. Centrifugation was performed with the chip at 800 rpm for 3 minutes, followed by drying thereof.

TABLE 2

| Component | Volume (μl) per reaction |
|---|---|
| nuclease-free water | 0.75 |
| 5X Transcription Buffer | 3.2 |
| 0.1M DTT | 0.6 |
| TP mix | 1 |
| T7 RNA Polymerase Blend | 0.21 |
| Cyanine 3-CTP | 0.24 |

### <2-4> Obtainment of Fluorescence Image

Hybridization images on the slide were scanned with Agilent C scanner (Agilent technologies, CA, USA). At that time, the green fluorescent image indicated the activity of gene expressed specifically in the control group, while the red fluorescent image indicated the activity of gene expressed specifically in the experimental group. In the meantime, the yellow fluorescent image indicated that there was no big difference in the expression between those genes respectively presented by red and green. To obtain gene expression rate, the scanned images were analyzed by using Agilent Feature Extraction 10.7.3.1 (Agilent technologies, CA, USA). The extracted data proceeded to normalization by using Agilent GeneSpring GX 11.5.1 (Agilent technologies, CA, USA) to analyze gene expression pattern of each gene. The marker gene for lower aliphatic saturated aldehydes was selected from the obtained data.

As a result, as shown in FIG. 2, among 42,000 genes existing on the oligo chip, the genes showing at least 1.5 fold higher expression (Cy5/Cy3 ratio) by lower aliphatic saturated aldehydes took 0.04% (16 genes out of 42,405 genes) and the genes showing lower expression took 0.01% (4 genes out of 42,405 genes) (Table 3 and FIG. 2).

TABLE 3

| | | | Median ratio of microarray | | | | | |
|---|---|---|---|---|---|---|---|---|
| Accession | | Gene | Propion | Butyl | Valer | | | |
| Number | Abbreviation | Name | aldehyde | aldehyde | aldehyde | hexanal | heptanal | octanal |
| NM_003782; | B3GALT4 | UDP- | 0.483 | 0.498 | 0.53 | 0.574 | 0.634 | 0.564 |
| SEQ. | | Gal:betaG1cNAc | | | | | | |
| ID. NO: | | beta 1,3- | | | | | | |
| 11 | | galactosyltransferase, | | | | | | |
| | | polypeptide 4 | | | | | | |
| NM_004321; | KIF1A | kinesin | 3.814 | 14.175 | 7.871 | 1.682 | 1.955 | 2.262 |
| SEQ. | | family | | | | | | |
| ID. NO: | | member 1A | | | | | | |
| 12 | | | | | | | | |
| NM_005771; | DHRS9 | dehydregenase/ | 2.988 | 2.353 | 2.088 | 1.654 | 2.157 | 3.057 |
| SEQ. | | reductase | | | | | | |
| ID. NO: | | (SDR | | | | | | |
| 13 | | family) | | | | | | |
| | | member 9 | | | | | | |
| NM_013447; | EMR2 | egf-like | 6.906 | 8.094 | 6.242 | 1.89 | 2.25 | 1.785 |
| SEQ. | | module | | | | | | |
| ID. NO: | | containing, | | | | | | |
| 14 | | mucin- | | | | | | |
| | | like, | | | | | | |
| | | hormone | | | | | | |
| | | receptor- | | | | | | |
| | | like 2 | | | | | | |
| NM_018689; | KIAA1199 | KIAA1199 | 4.454 | 1.733 | 3.122 | 1.736 | 2.815 | 2.294 |
| SEQ. | | | | | | | | |
| ID. NO: | | | | | | | | |
| 15 | | | | | | | | |

### Example 3

### Quantitative Real-Time RT-PCR

To investigate and quantify the expressions of 5 different genes confirmed to be expressed specifically by lower aliphatic saturated aldehydes [Genebank accession number NM_{—}003782 (B3GALT4, UDP-Gal:betaGlcNAc beta 1,3-galactosyltransferase, polypeptide 4; SEQ. ID. NO: 11), Genebank accession number NM_{—}004321 (KIF1A, kinesin family member 1A; SEQ. ID. NO: 12), Genebank accession number NM_{—}005771 (DHRS9, dehydrogenase/reductase (SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15)], selected in Example 2 among many genes demonstrating up- or down-regulation specifically by lower aliphatic saturated aldehydes, quantitative real-time RT-PCR was performed using My IQ real-time PCR machine (Bio-rad, USA).

Particularly, cDNA was synthesized by performing reverse transcription by using oligo dT primer and Superscript kit (Omniscipt™ kit, Qiagen, Co., USA). 0.2 μl of the synthesized cDNA was mixed with 3.8 μl of water, 0.5 μl of sense primer, 0.5 μl of anti-sense primer, and 5 μl of SYBR Green I staining supermix (Bio-rad, USA), which was loaded in PCR tube, followed by reaction in My IQ real-time PCR machine designed to execute reaction as follows: step 1: 95° C., 3 minutes; step (45 cycles): step 2-1: 95° C., 10 seconds; step 2-2: KIF1A—57° C., B3GALT4, DHRS9, EMR2, KIAA1199—65° C., 45 seconds; step 3: 95° C., 1 minute; step 4: 55° C., 1 minute; step 5 (80 cycles): 55° C., 10 seconds. The PCR product was stained with SYBR Green I (Bio-rad, USA) to quantify thereof. SYBR Green I staining is the method taking advantage of intercalating with double-stranded DNA, and thus the more double-stranded DNA is produced, the stronger the fluorescence intensity is obtained PCR. Primers for the target gene used for PCR and the endogenous control (RPLPO) were added to SYBR Green master-mix, followed by PCR. Primer optimization was performed to determine a proper concentration. The synthesized cDNA was mixed with each primer listed in Table 5, to which SYBR Green master-mix was added. Then, PCR was performed and the result was analyzed by using quantitative software.

As a result, expression patterns of those genes [Genebank accession number NM_{—}003782 (B3GALT4, UDP-Gal:betaGlcNAc beta 1,3-galactosyltransferase, polypeptide 4; SEQ. ID. NO: 11), Genebank accession number NM_{—}004321 (KIF1A, kinesin family member 1A; SEQ. ID. NO: 12), Genebank accession number NM_{—}005771 (DHRS9, dehydrogenase/reductase (SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15)] were very similar to the results of microarray chip analysis of Example <2-4>.

TABLE 5

| Genbank | | | |
|---|---|---|---|
| Accession | Gene | PCR Primer Sequence | |
| Number | Name | (5′->3′) | |
| NM_003782; | B3GALT4 | Sense | TCCGGAGAACCTGAACCAGAGAAA |
| SEQ. ID. | | (SEQ. ID. NO: | |
| NO: 11 | | 1) | |
| | | Antisense | TCTCCCAGCAAGAATAGCGTCTGT |
| | | (SEQ. ID. NO: | |
| | | 2) | |
| NM_004321; | KIF1A | Sense | TTCTACCACGTCCAGAACATCGCA |
| SEQ. ID. | | (SEQ. ID. NO: | |
| NO: 12 | | 3) | |
| | | Antisense | TGGTAGTGGCCAAAGACCTCGAAA |
| | | (SEQ. ID. NO: | |
| | | 4) | |
| NM_005771; | DHRS9 | Sense | ACACTGTTAGACCAAGGGCACAGA |
| SEQ. ID. | | (SEQ. ID. NO: | |
| NO: 13 | | 5) | |
| | | Antisense | ATTTGATGCAGCGTGTAGCCAAGG |
| | | (SEQ. ID. NO: | |
| | | 6) | |
| NM_013447; | EMR2 | Sense | AACCTCGAAGATGGGTATGGCACA |
| SEQ. ID. | | (SEQ. ID. NO: | |
| NO: 14 | | 7) | |
| | | Antisense | AGATGGTTCTCACTCTGTTGCCCA |
| | | (SEQ. ID. NO: | |
| | | 8) | |
| NM_018689; | KIAA1199 | Sense | TGGGTGTCTGAACAGCTATTGGGT |
| SEQ. ID. | | (SEQ. ID. NO: | |
| NO: 15 | | 9) | |
| | | Antisense | ACTCTGGTGCCTCGTGCAAGATTA |
| | | (SEQ. ID. NO: | |
| | | 10) | |

### INDUSTRIAL APPLICABILITY

As explained hereinbefore, the microarray chip or the kit developed in this invention can be effectively used for the monitoring of 6 kinds of lower aliphatic saturated aldehydes (propionaldehyde, butylaldehyde, valeraldehyde, hexanal, heptanal, and octanal) and for the evaluation of risk of the said lower aliphatic saturated aldehydes.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

What is claimed is:

1. A method for the identification of exposure to lower aliphatic saturated aldehydes (propionaldehyde, butylaldehyde, valeraldehyde, hexanal, heptanal, and octanal) comprising the following steps:
1) measuring expression levels of genes of:
Genebank accession number NM_{—}003782 (B3GALT4, UDP-Gal:betaGlcNAc beta 1,3-galactosyltransferase, polypeptide 4; SEQ. ID. NO: 11), Genebank accession number NM_{—}004321 (KIF1A, kinesin family member 1A; SEQ. ID. NO: 12), Genebank accession number NM_{—}005771 (DHRS9, dehydrogenase/reductase (SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15), on somatic cells separated from both an experimental group suspected with lower aliphatic saturated aldehydes exposure and a normal control group;
2) screening a subject with increased or decreased expression level by comparing the expression level of the experimental group of step 1) with that of the control group; and
3) determining the screened object of step 2) to be exposed to lower aliphatic saturated aldehydes.

2. The method for the identification of exposure to lower aliphatic saturated aldehydes according to claim 1, wherein the somatic cells of step 1) are characteristically human lung cells or human lung cancer tissue derived cells.

3. The method for the identification of specific exposure to lower aliphatic saturated aldehydes according to claim 2, wherein the human lung cancer tissue derived cells are A549.

4. The method for the identification of specific exposure to lower aliphatic saturated aldehydes according to claim 1, wherein the comparing the expression level in step 1) is performed at the level of gene or protein.

5. The method for the identification of specific exposure to lower aliphatic saturated aldehydes according to claim 4, wherein the comparing at the level of gene uses any one selected from a group consisting of RT-PCR, competitive RT-PCR, real-time RT-PCR, RNase protection assay, Northern blotting, and DNA chip.

6. The method for the identification of specific exposure to lower aliphatic saturated aldehydes according to claim 4, wherein the comparing at the level of protein uses microarray or ELISA.

7. The method for the identification of specific exposure to lower aliphatic saturated aldehydes according to claim 1, wherein the expression level of the genes of Genebank accession number NM_{—}004321 (KIF1A, kinesin family member 1A; SEQ. ID. NO: 12), Genebank accession number NM_{—}005771 (DHRS9, dehydrogenase/reductase (SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15) is up-regulated when exposed to lower aliphatic saturated aldehydes.

8. The method for the identification of specific exposure to lower aliphatic saturated aldehydes according to claim 1, wherein the expression level of the gene of Genebank accession number NM_{—}003782 (B3GALT4, UDP-Gal:betaGlcNAc beta 1,3-galactosyltransferase, polypeptide 4; SEQ. ID. NO: 11) is down-regulated when exposed to lower aliphatic saturated aldehydes.

9. A method for the identification of exposure to lower aliphatic saturated aldehydes (propionaldehyde, butylaldehyde, valeraldehyde, hexanal, heptanal, and octanal) comprising the following steps:
1) extracting RNA from somatic cells obtained from both the experimental group highly suspected with lower aliphatic saturated aldehyde exposure and the normal control group;
2) synthesizing cDNA from the RNA extracted from both the experimental group and the control group of step 1), followed by labeling with different fluorescent materials;
3) hybridizing each cDNA labeled with different fluorescent materials of step 2) with the DNA microarray chip on which nucleic acid sequences of one or more genes selected from the below group or their complementary strand molecules are integrated:
Genebank accession number NM_{—}003782 (B3GALT4, UDP-Gal:betaGlcNAc beta 1,3-galactosyltransferase, polypeptide 4; SEQ. ID. NO: 11), Genebank accession number NM_{—}004321 (KIF1A, kinesin family member 1A; SEQ. ID. NO: 12), Genebank accession number NM_{—}005771 (DHRS9, dehydrogenase/reductase (SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15);
4) analyzing the reacted DNA microarray chip; and
5) confirming the exposure to lower aliphatic saturated aldehydes by comparing the expressions of the genes integrated on the DNA microarray chip with those of the control based on the data analyzed.

10. The method for the identification of exposure to lower aliphatic saturated aldehydes according to claim 9, wherein the somatic cell of step 1) is characteristically human lung cell or human lung cancer tissue derived cell.

11. The method for the identification of specific exposure to lower aliphatic saturated aldehydes according to claim 10, wherein the human lung cancer tissue derived cell is A549.

12. The method for the identification of exposure to lower aliphatic saturated aldehydes according to claim 9, wherein the fluorescent material of step 3) is selected from the group consisting of Cy3, Cy5, poly L-lysine-fluorescein isothiocyanate (FITC), RITC (rhodamine-B-isothiocyanate), and rhodamine.

13. A method for the identification of exposure to lower aliphatic saturated aldehydes (propionaldehyde, butylaldehyde, valeraldehyde, hexanal, heptanal, and octanal) comprising the following steps:
1) extracting RNA from somatic cells obtained from both the experimental group highly suspected with lower aliphatic saturated aldehyde exposure and the normal control group;
2) performing real-time RT-PCR (real-time reverse transcript polymerase chain reaction) with the obtained RNA using the primer sets complementary to the below genes and able to amplify them as well:
Genebank accession number NM_{—}003782 (B3GALT4, UDP-Gal:betaGlcNAc beta 1,3-galactosyltransferase, polypeptide 4; SEQ. ID. NO: 11), Genebank accession number NM_{—}004321 (KIF1A, kinesin family member 1A; SEQ. ID. NO: 12), Genebank accession number NM_{—}005771 (DHRS9, dehydrogenase/reductase (SDR family) member 9; SEQ. ID. NO: 13), Genebank accession number NM_{—}013447 (EMR2, egf-like module containing, mucin-like, hormone receptor-like 2; SEQ. ID. NO: 14), and Genebank accession number NM_{—}018689 (KIAA1199, KIAA1199; SEQ. ID. NO: 15); and
3) confirming the expression by comparing the gene product obtained in step 2) with that of the control.

14. The method for the identification of exposure to lower aliphatic saturated aldehydes according to claim 13, wherein the primer set of step 2) is characteristically composed of the forward primer and the reverse primer in the length of 18-30 mer that is able to amplify the gene of step 2).

15. The method for the identification of exposure to lower aliphatic saturated aldehydes according to claim 13, wherein the primer set of step 2) is selected from the group consisting of the following primer set 1˜primer set 5:
Primer set 1: forward primer represented by SEQ. ID. NO: 1 and reverse primer represented by SEQ. ID. NO: 2;
Primer set 2: forward primer represented by SEQ. ID. NO: 3 and reverse primer represented by SEQ. ID. NO: 4;
Primer set 3: forward primer represented by SEQ. ID. NO: 5 and reverse primer represented by SEQ. ID. NO: 6;
Primer set 4: forward primer represented by SEQ. ID. NO: 7 and reverse primer represented by SEQ. ID. NO: 8; and
Primer set 5: forward primer represented by SEQ. ID. NO: 9 and reverse primer represented by SEQ. ID. NO: 10.
